(19) United States
(12) Patent Application Publication
(10) Pub. No.: US 2007/0244492 A1
(43) Pub. Date: Oct. 18, 2007

(54) Fallopian tube intra-luminal splint for reanastomosis

(76) Inventors: Bo Young Suh, Los Angeles, CA (US); Fu-Nan Wang, Hacienda Heights, CA (US); Winston Zonh Ho, Hacienda Heights, CA (US)

Correspondence Address: Winston Z. Ho, 14541 Langhill Drive, Hacienda Heights, CA, 91745, US

(21) Appl. No.: 11/345,728

(22) Filed: Feb. 2, 2006

(51) Int. Cl. A61B 17/10

(52) U.S. Cl. 606/139

(57) ABSTRACT

The tubal splint is designed to fit the inner side of a tube, such as fallopian tubes, and can be slided in through the fimbriated end (outer side of entrance toward the uterus) prior to the reanastomosis. This invention is to provide a simple method for tubal reanastomosis suturing operation. By utilizing the splint, both ends of the tube can yield the best prognostic position for the suturing. This invention is to provide a tubal splint so that the stitches can be placed easily and appropriately on the right spots when both ends are arranged in proximity.

### FIELD OF THE INVENTION

The present invention is generally related to a method and apparatus for tubal reanastomosis with micro-suturing. A tubal splint is designed to fit the inner side of fallopian tube, and can be slided in through the fimbriated end prior to the reanastomosis. The tubal splint brings the edges of both tubal ends at the same levels; therefore the stitches can be placed easily and appropriately on the right spots when both ends are arranged in proximity.

### BACKGROUND OF THE INVENTION

Although the availability of assisted reproductive technology has reduced the need for reconstructive surgery in sub-fertile women, when fertility surgery is indicated, open laparotomy is usually carried out. The reversal of tubal ligations may be beneficial than an assisted reproductive technology for women in less than 35 year-old for their future conceptions. In view of the factors that the physicians reluctant to practice comfortably are: 1) the surgery is taking place under the magnified microscopic field; 2) the suture is a hair thin synthetic threads; 3) it is not easy to work under the magnified area unless the surgeons are specifically trained in this area; 4) the duration of surgery takes about two to three hours in experienced hands. However, it may take many more hours in inexperienced hands; 5) to equip the microscope in the operating room is costly (>$30,000); and 6) the failure rate is high after the procedures. Therefore, it would be beneficial to both the physicians and patients if there are ways to complete the procedure in a simple manner without expensive equipment and shorten the duration of the surgery with a better prognosis. The method and apparatus of fallopian tube reanastomosis procedures with the aid of fallopian tube intra-luminal splint (FATILS) is disclosed in this invention.

Indications for tubal anastomosis include reversal of sterilization, midtubal block secondary to pathology, tubal occlusion from ectopic pregnancy, and salpingitis isthmica nodosa. The goal is to remove abnormal tissue and reapproximate the healthy tubal segments with as little adhesion formation as possible. The technique involves micro-suturing using 6-0 to 10-0 sutures.

Sterilization reversal, although not always successful, is the most successful surgical reconstructive procedure for improving fertility. Factors that may influence the success rate of tubal reanastomosis include the age of the patient, time from sterilization, and sterilization technique. In one large series study, for example, pregnancy rates among women aged 15 to 30 years, 30 to 33 years, and 34 to 49 years were 73, 64, and 46 percent, respectively. Most pregnancies occurred within two years after reversal. Of interest, 23 percent of patients subsequently underwent another sterilization. In another series study, tubal anastomosis resulted in live births in 41 percent of women with a previous electro-cautery procedure, 50 percent of those who had a Pomeroy tubal ligation, 75 percent of women with rings, and 84 percent of those with clips. Tubal length is another important factor in successful reversal. The pregnancy rate after tubal anastomosis is 75 percent in women with tubal length of 4 cm or more, but only 19 percent in those with shorter tubes. Pregnancy rates after laparoscopic tubal anastomosis and conventional microsurgical anastomosis are equivalent, 80 to 81 percent 12 months after surgery. Ectopic pregnancy rates are also similar at 2.5 to 2.8 percent. The disadvantage of laparoscopic tubal anastomosis is that it is more demanding technically than an open microsurgical procedure.

U.S. Pat. No. 5,879,371 to Gardiner et al., entire contents of which are incorporated herein by reference, discloses a surgical fastener in the form of a ferruled loop, instrument and method are provided for constructing a graft to artery anastomosis and other soft tissue anastomoses, particularly by minimally invasive (or endoscopic) surgery. The ferruled loop fastener is comprised of a needle, a suture and a ferrule. The needle and suture member allow this fastener to be sewn through, for example, a graft and artery to be joined. The ferrule member allows a ferruled loop to be formed without knotting the suture member, and that serves as a suture stitch to fasten the graft and artery together. The instrument holds the needle and ferrule at its distal end, and controls in the handle allow manipulation of the needle and controllable application of the fastener. The method employs the ferruled loop and instruments to join soft tissues and to construct graft to artery anastomoses.

U.S. Pat. No. 4,553,542 to Schenck et al., entire contents of which are incorporated herein by reference, discloses encircling devices by which anatomical structures, such as blood vessels, fallopian tubes, ureters, vas deferens and outer nerve sheaths are anastomosed. Such an encircling device provides an opening that receives an end of a tubular anatomical structure having a prepared opening, and the tubular structure is anastomosed to a second anatomical structure having a prepared opening by tethering the two structures to the encircling device holding the structures under radial stress in apposition to each other to form a fluid-tight peripheral seal around their openings.

U.S. Pat. No. 4,553,542 to Schenck et al. further discloses a stent or support for use in the connection or anastomosis of severed vessels to support and seal the anastomotic site. The stent includes substantially cylindrical sections separated by a tapered transitional region. The cylindrical sections are provided with flanges that define tapered sealing surfaces. The dimensions of the two sections are selected to correspond with the diameter of the portions of the vessel to be supported. The stent is preferably made of polyglycolic acid and the dimensions of the stent are selected to provide optimal support and sealing characteristics with a minimum of damage to the epithelial lining of the vas deferens. In three preferred applications, the stent is used in anastomosis of the severed ends of a vas deferens, a fallopian tube, and a blood vessel.

Although many prior art patents are related to anastomosis operation, none of them discloses an apparatus and method which is designed with an intra-luminal splint which can be inserted through the fimbrial orifice for fallopian tube suturing.

### SUMMARY OF THE INVENTION

Accordantly, the invention relates to a method for fallopian tube micro-suturing for the reanastomosis of the fallopian tube comprising the steps of (a) sliding a tubal splint entering through the fimbrial orifice, passing the distal portion of the ligated fallopian tube, to the proximal entrance of the ligated tube possibly reaching to the thinnest portion of the fallopian tube; (b) bringing proximal and distal portions of the tubes in proximity to yield the best prognostic position for the suturing; (c) placing sutures at various positions of tubal muscular layer of the fallopian tube; and (d) withdrawing the tubal splint from the fimbrial orifice.

Another object of this invention is to provide a tubal splint designed for the tubal reanastomosis. The tubal splint comprising a cylindrical tube having one end of the cylindrical tube is small enough to fit in the inner opening of the fallopian tube and the distal portion of the cylindrical tube can fit snuggly the end portion of the ligated fallopian tube; and the cylindrical tube is long enough to be slided through the area for suturing.

Another object of this invention is to provide a tubal splint to bring the edges of both tubal ends at the same levels with sutures. It is not an easy procedure especially when both ends are in different diameters. By utilizing the splint, both ends of the tube can yield the best prognostic position for suturing.

Another object of this invention is to provide a tubal splint, wherein the stitches can be placed easily and appropriately on the right spots when both ends are arranged in proximity.

Another object of this invention is to provide a tubal splint, wherein the surgery time can be shortened to less than two hours because of the easiness to place the sutures.

The present tubal splint device has the advantages of simple, real time, and easy operation. The device provides rapid and accurate results to assist clinician's suturing operation. It should be understood, however, that the detailed description and specific examples, while indicating preferred embodiments of the present invention, are given by way of illustration and not of limitation. Further, as will become apparent to those skilled in the art, the teaching of the present invention can be applied to medical devices for performing suturing at a variety of body parts.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a perspective view of a fallopian tube intra-luminal splint (a) the longitudinal view and (b) the cross-section view.

FIG. 2 shows a fallopian tube intra-luminal splint that is inserted into the fallopian tube through the fimbrial orifice.

FIG. 3 shows a fallopian tube intra-luminal splint that is used to assist micro suturing.

### DETAILED DESCRIPTION OF EXEMPLARY EMBODIMENTS

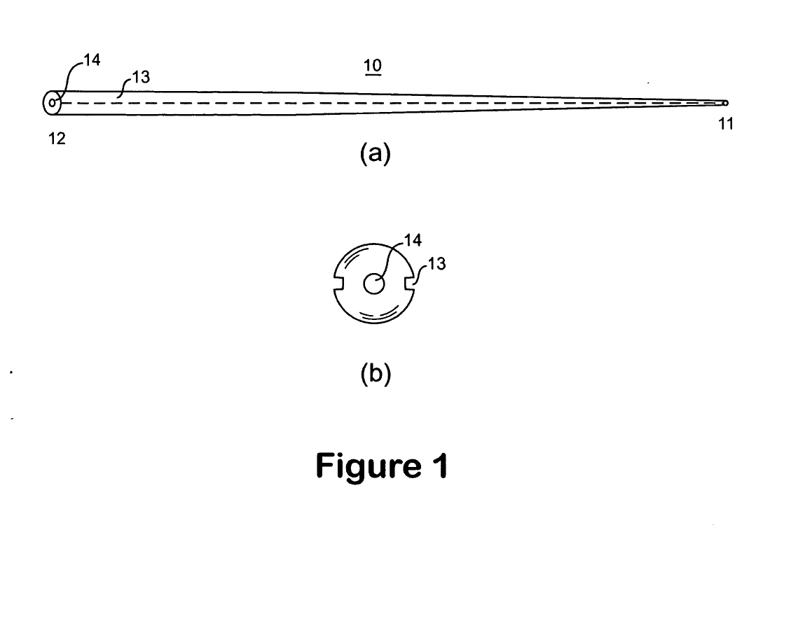

The preferred embodiments of the present invention described below relate particularly to the properties of the tubal splint. There are two types of splints, of which the first type is in blue color and the second type is a clear color with semi-transparent characteristic. The splint is a flexible cylindrical tube 10 and is configured soft enough not to perforate or penetrate the soft tissues. In one embodiment, silicone-based plastics are suitable for this application. The rigidity of the cylindrical tube is critical, it should be soft enough not to perforate the tubal wall, and rigid enough for sliding through the whole fallopian tube. In some embodiment, the “rigidity” also refers to pushability of the cylindrical tube. The tip 11 of the splint is 0.3-3.0 mm in diameter and round shape as shown in FIG. 1. In an alternate embodiment, the cross-section of the splint may be oval shape, circular shape, semi-circular shape, or other shape.

Certain portion of the fallopian tube, such as isthmic section, has an intro-channel of 0.5-3 mm in diameter. Therefore, the smaller end or tip of the tubal splint should be able to slide through this portion of the fallopian tube. The larger end 12, proximal end or handle of the splint is about 6 mm or larger in diameter and a total length from the tip to the handle is approximately 30 cm. The diameter from the tip to the handle runs from about 0.3 mm to 6 mm. The tip of the splint is sized and designed to be small enough to fit in the inner opening of the tube (connected to the uterus) and that the distal portion of the splint can fit snuggly the outer portion of the tube. The splint consists of multiple tranversed grooves 13 in the area of 9 cm from the end of handle. There are two longitudinal grooves on each side of the splint starting from about 7 cm from the thinnest part to the end of handle to drain blood during surgery. In one embodiment, the tubal splint further comprises a hollow channel 14 along the center core for liquid dispensing. When the splint is used in conjunction with a syringe, various liquids, or dye solutions can be injected and dispensed into the fallopian tube.

Pre-Surgery Preparation

Infertile patient with obstruction of fallopian tube, due to adhesion or blockage, would be taken to the operating room for epidural or general anesthesia. She would then be prepared and draped in a normal sterile fashion in the dorsal spine position. A transverse or vertical skin incision dependent on the previous scar, approximately two finger breadths above the symphysis pubis, would be made with the scalpel and carried down to the fascia, which would be incised in the midline and the incision will be extended laterally with the curved Mayo scissors. The superior aspect of the fascial incision would then be grasped with the Kocher clamps, elevated, and the underlying rectus muscles would be dissected off carefully. The rectus muscles are then separated in the midline, and the peritoneum identified, tented up, and entered with Metzenbaum scissors. To prevent possible bladder injury during the procedures, the peritoneal incision would be extended superiorly and inferiorly under a direct good visualization. After the inspection of the pelvis including the fallopian tubes, ovaries, round ligaments, adjacent bowels, and possibly related and adjacent adhesions the procedure will be proceeded.

Suturing with the Aid of Tubal Splint

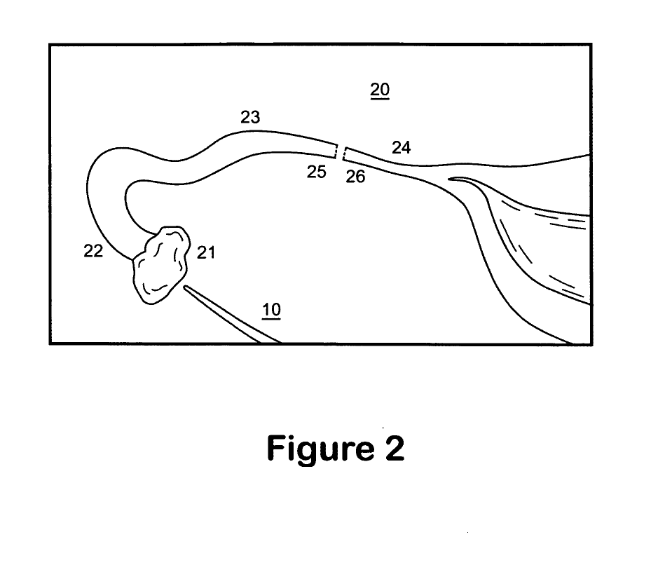

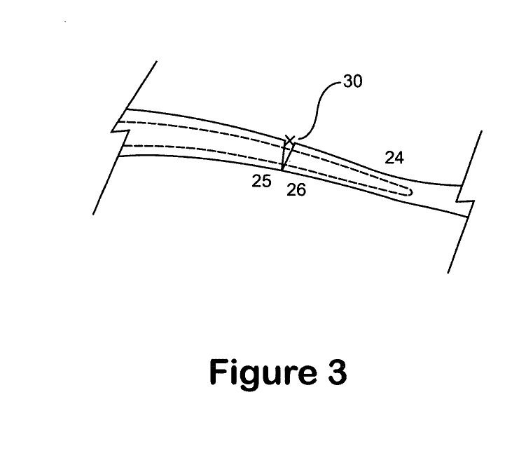

After identification of the pathologically blocked area of the fallopian tube, a segmental resection is carried out by shaving the proximal and followed by the distal portion of the tubes until the blue dye spurts. Then, mesosalpinx, is vertically cut to give more room during the procedure. The proximal portion 25 of the tube is shaved until the healthy lumen of the tube is identified. Then the same procedure is carried out in the distal portion 26 of the tube. The tube splint 10 is ready to be inserted through the fimbrial orifice 21 (the most outer portion of the tube) as shown in FIG. 2. Holding the FATILS handle, the tip or the smaller end is slided through the fimbriated end (outer side of entrance toward the uterus). Then pass the distal portion of the shaved tube to the proximal entrance of the shaved tube possibly reaching to the thinnest portion of the tube. After insertion, the splint is adjusted so to fit snugly in both the proximal and the distal portion of the tube are, which could be dilated maximally according to the thickness of splint and this fitting would provides the best prognostic position for the reanastomosis. As shown in FIG. 3, thus the proximal and distal portions of the tube are brought in proximity by placing sutures 30 at 12, 3, 6, and 9 o'clock positions of tubal muscular layer using 6-0 or 7-0 prolene, or absorbable suture, e.g., Dexon or Vicryl, flowed by the same sutures in the same positions of the serosal layer. This procedure can be performed with a microscopy, loops or without a microscopy. Upon the completion of the surgery, the splint is withdrawn from the fimbrial end. Alternatively, the tubal splint may remain in place, with some portion of the tube extends outside of patient's lower abdomen, to prevent possible further re-adhesions. After 48-72 hours, the tubal splint can be removed away from the abdomen externally.

The FATILS, in addition for suturing aid, can also be used for blockage examination. When the tubal splint is inserted gradually from the ostium of the fimbrial orifice 21, then into the infundibular section 22, then into the ampullary portion 23 or isthmus portion 24, the exact location of the pathological blockage can be identified when the splint is obstructed. Furthermore, in an effort to verify that the fallopian tube is patent, a volume of 0.5 mL normal saline or Methylene Blue dye would be injected through the central pore or lumen of the FATILS with a syringe. By examining the liquid flow in the fallopian tube, the patency of the operated fallopian tube would be secured. Any leakage or blockage would indicate the failure of the operation.

From the foregoing, it should now be appreciated that a fallopian tubal splint apparatus and methods for tubal reanastomosis with micro-suturing has been disclosed. It is also generally applicable for suturing in many parts of the body. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

1. A tubal splint for fallopian tube micro-suturing comprising an elongate cylindrical tube having a distal end, a proximal end, and a distal portion, wherein said distal end of said cylindrical tube is small enough to fit in an inner opening of the fallopian tube and the distal portion of said cylindrical tube fits snuggly a portion of the fallopian tube; and said cylindrical tube is long enough to be slided through an area of said opening for micro-suturing.

2. The tubal splint defined in claim 1, wherein the distal end of said cylindrical tube is about 0.3-3.0 mm in diameter.

3. The tubal splint defined in claim 1, wherein a cross-section of said distal end is a round shape or oval shape.

4. The tubal splint defined in claim 1, wherein the proximal end of said cylindrical tube is between about 3.0 and 10.0 mm in diameter.

5. The tubal splint defined in claim 1, wherein said tubal splint is about 5.0-30 cm in length.

6. The tubal splint defined in claim 1, wherein said tubal splint is made of silicone plastics.

7. The tubal splint defined in claim 1, wherein said cylindrical tube further comprises at lease one groove along an outer core of said cylindrical tube for fluid drainage.

8. The tubal splint defined in claim 1, wherein said cylindrical tube further comprises a hollow channel along a center core of said cylindrical tube for liquid dispensing.

9. A method for fallopian tube micro-suturing for reanastomosis of a ligated tube comprising the steps of: (a) sliding a tubal splint entering through a fimbrial orifice, passing a distal portion of the ligated tube, to a proximal entrance of the ligated tube substantially reaching a thinnest portion of the fallopian tube; (b) bringing proximal and distal portions of the ligated tubes in proximity to yield a best prognostic position for the micro-suturing; (c) placing sutures at various positions of a tubal muscular layer of said fallopian tube; and (d) withdrawing said tubal splint from the fimbrial orifice.

10. The method defined in claim 9, wherein a distal end of said tubal splint is about 0.3-3.0 mm in diameter and round shape.

11. The method defined in claim 9, wherein a proximal end of said tubal splint is between about 3.0 and 10.0 mm in diameter.

12. The method defined in claim 9, wherein said tubal splint is about 5.0-30 cm in length.

13. The method defined in claim 9, wherein said tubal splint is made of silicone plastics.
